(19) United States
(12) Patent Application Publication
(10) Pub. No.: US 2003/0041337 A1
(43) Pub. Date: Feb. 27, 2003

(54) Transgenic animals containing a dominant negative mutant form of the p85 subunit of PI-3 kinase

(76) Inventors: E. Michael Gibbs, Oakdale, CT (US); John D. McNeish, Mystic, CT (US)

Correspondence Address: Gregg C. Benson, Pfizer Inc., Patent Department, MS 4159, Eastern Point Road, Groton, CT, 06340, US

(21) Appl. No.: 10/081,980

(22) Filed: Feb. 20, 2002

Related U.S. Application Data
(60) Provisional application No. 60/270,014, filed on Feb. 20, 2001.

(51) Int. Cl. A01K 67/027

(52) U.S. Cl. 800/4; 800/18; 800/14

(57) ABSTRACT

The present invention provides a genetically-modified, non-human mammal having an altered body fat composition, wherein said mammal comprises an exogenous mutant p85 PI3-K gene, wherein the expression of said gene is driven by an insulin responsive cell specific promoter. The present invention also relates to transgenic cell lines containing the same mutant gene, as well as methods using both animals and/or cells.

### CROSSREFERENCE TO RELATED APPLICATION

This application claims priority from the U.S. Provisional Patent Application No. 60/270,014 filed Dec. 21, 2001, the benefit which is hereby claimed under 37 C.F.R. §1.78(a)(3).

### FIELD OF THE INVENTION

The present invention relates to transgenic animals containing a dominant negative mutant form of the regulatory p85 subunit of phosphatidylinositol 3-kinase. Surprisingly, rather than being a diabetic phenotype, these animals exhibit an altered body fat composition, having a marked increase in body fat coupled with a corresponding decrease in lean muscle mass. The present invention also relates to transgenic cell lines containing the same mutant gene, as well as methods for using both animals and/or cells.

### BACKGROUND OF THE INVENTION

Many growth factors and hormones such as nerve growth factor, platelet derived growth factor, epidermal growth factor, and insulin mediate their signals through interactions with cell surface tyrosine kinase receptors. The transduction of extracellular signals across the membrane, initiated by ligand binding, leads to the propagation of multiple signaling events which ultimately control target biochemical pathways within the cell.

The phosphatidylinositol 3-kinases (PI3Ks) represent a ubiquitous family of heterodimeric lipid kinases that are found in association with the cytoplasmic domain of hormone and growth factor receptors and oncogene products. PI3Ks act as downstream effectors of these receptors, are recruited upon receptor stimulation and mediate the activation of second messenger signaling pathways through the production of phosphorylated derivatives of inositol (Fry, Biochim. Biophys. Acta., 1226:237-68 (1994)).

PI3Ks have been implicated in many cellular activities including growth factor mediated cell transformation, mitogenesis, protein trafficking, cell survival and proliferation, DNA synthesis, apoptosis, neurite outgrowth, and insulin-stimulated glucose transport (reviewed in Fry, Id.).

The PI3 kinase enzyme heterodimers consist of a 110 kD (p110) catalytic subunit associated with an 85 kD (p85) regulatory subunit and it is through the SH2 domains of the p85 subunit that the enzyme associates with the membrane-bound receptors (Escobedo et al., Cell, 65:75-82 (1991); Skolnik et al., Cell, 65:83-90 (1991)).

PI3K p85 (also known as GRBI and PIK3R1) was initially isolated from bovine brain and shown to exist in two forms, α and β. In these studies p85 isoforms were shown to bind to and act as substrates for tyrosine-phosphorylated receptor kinases and the polyoma virus middle T antigen complex (Otsu et al., Cell, 65:91-104 (1991)). Since then the p85 subunit has been further characterized and shown to interact with a diverse group of proteins including tyrosine kinase receptors such as the erythropoietin receptor, the PDGR-β receptor, and Tie2, an endothelium-specific receptor involved in vascular development and tumor angiogenesis (Escobedo et al., Cell, 65:75-82 (1991); He et al., Blood, 82:3530-38 (1993); Kontos et al., Mol. Cell. Biol., 18:4131-40 (1998)). It also interacts with focal adhesion kinase (FAK), a cytoplasmic tyrosine kinase involved in integrin signaling and is thought to be a substrate and effector of FAK. The p85 subunit also interacts with profilin, an actin binding protein that facilitates actin polymerization (Bhargavi et al., Biochem. Mol. Biol. Int., 46:241-248 (1998); Chen and Guan, PNAS, 91:10148-52 (1994)) and the p85/profilin complex inhibits actin polymerization.

Recently, a truncated form of the PI3K p85 subunit was isolated (Jimenez et al., Embo J., 17:743-53 (1998)). This form includes the first 571 amino acids of the wild type (encoded by nucleotides 43-1755 of Genbank Act. No. M61906) linked to a region that is conserved in the eph tyrosine kinase receptor family. This truncation was shown to induce the constitutive activation of PI3 kinase and contribute to cellular transformation of mammalian fibroblasts.

Several chemically distinct inhibitors for PI3 kinases are reported in the literature. These include wortmannin, a fungal metabolite (Ui et al., Trends Biochem. Sci., 20:303-07 (1995)), demethoxyviridin, an antifungal agent (Woscholski et al., FEBS Lett., 342:109-14 (1994)), and quercetin and LY294002, two related chromones (Vlahos et al., J. Biol. Chem., 269:5241-48 (1994)). However, these inhibitors primarily target the p110 subunit.

### SUMMARY OF THE INVENTION

Insulin activates PI3-K by stimulating the tyrosine phosphorylation of IRS-1 and IRS-2 which then bind to p85 via SH2 domains and results in the rapid activation of the p110 catalytic domain. Thus, overexpression of mutant p85 was expected to lead to a situation in which PI3-K is not stimulatable by insulin because most of the phospho-IRS-1 and phospho-IRS-2, which normally binds to p85 and activates PI3-K, will be complexed with mutant p85 molecules.

Thus, to achieve the present invention, a DNA expression vector was produced containing a truncated mouse p85 gene encoding a protein that contains the two SH2 domains, but lacks the inter-SH2 domain required for binding p110. This mutant gene was placed under the control of the muscle and fat cell specific Glut4 promoter/enhancer (see FIG. 1). This promoter consists of 2.1 kb of 5′ Glut4 flanking DNA that contains the cis-acting DNA sequences required for tissue-specific expression of the Glut4 protein (Olson et al., J. Biol. Chem., 268:9839-46 (1993)). The Glut4 promoter/enhancer element has been shown to direct high level gene expression of the Glut4 protein (Id.; and Gibbs et al., J. Clin. Invest., 95:1512-18 (1995)) as well as a chloramphenicol acetyltransferase (CAT) reporter gene (Olson, Id.). Successful expression with the CAT construct indicates that elements within the Glut4 coding sequence are not required for promoter activity. Thus, transgenic animals and cells may be produced containing this DNA construct.

In a first aspect, the present invention provides a genetically-modified, non-human mammal having an altered body fat composition, wherein said mammal comprises an exogenous mutant p85 PI3-K gene, wherein the expression of said gene is driven by an insulin responsive cell specific promoter. In a preferred embodiment, the mammal is a mouse comprising an exogenous mutant p85 PI3-K gene having the amino acid sequence of SEQ ID NO: 1, wherein the expression of said gene is driven by the Glut4 promoter.

In a second aspect, the present invention provides a genetically-modified animal cell, wherein said cell comprises an exogenous mutant p85 PI3-K gene, wherein the expression of said gene is driven by an insulin responsive cell specific promoter. In a preferred embodiment, the cell is a mouse cell comprising an exogenous mutant p85 PI3-K gene having the amino acid sequence of SEQ ID NO: 1, wherein the expression of said gene is driven by the Glut4 promoter.

In a third aspect, the present invention provides a method of identifying an agent that modulates the body fat composition of a mammal, said method comprising the steps of comparing the body fat compositions of a test animal to a control animal, wherein said agent has been administered to said test animal, further wherein each of said test and control animals is a genetically-modified, non-human mammal having an increased body fat composition, wherein said mammal comprises an exogenous mutant p85 PI3-K gene, wherein the expression of said gene is driven by an insulin responsive cell specific promoter, and identifying as a modulating agent the agent that has a statistically significant effect on the body fat composition of the test animal.

In a fourth aspect, the present invention provides a DNA expression construct for expressing a mutant p85 PI3-K gene, said construct comprising a mutant p85 PI3-K gene, wherein said gene encodes a p85 PI3-K protein whose inter-SH2 domain is unable to functionally interact with the p110 subunit of PI3-K, and an insulin responsive cell specific promoter that controls the expression of said gene. In a preferred embodiment, the construct comprises a mutant p85 PI3-K gene, wherein said gene encodes a mutant p85 PI3-K protein having the amino acid sequence of SEQ ID NO: 1, and a Glut4 promoter that controls the expression of said gene.

### DESCRIPTION OF THE DRAWINGS

FIG. 1 provides a representation of the mutant p85 dominant negative transgenic DNA construct used to create the mutant cells and mammal lines of the present invention. The nucleotide sequence of a preferred embodiment of this DNA construct is seen in SEQ ID NO: 2.

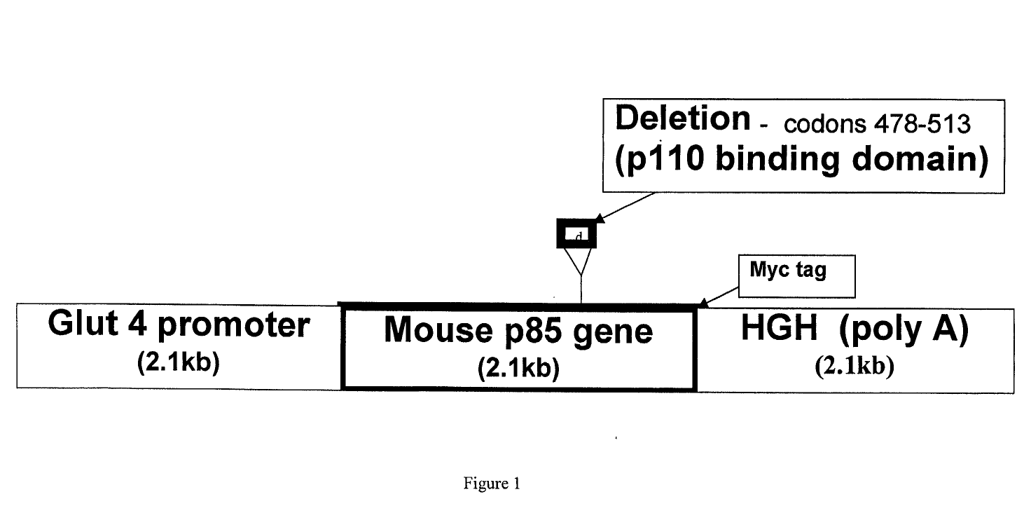

FIG. 2 provides a table showing a comparison of the fat, lean muscle, and total weight compositions of wild-type mice and mice produced according to the present invention.

FIG. 3 provides a graphical presentation of the body fat percentages over time of wild-type mice and mice produced according to the present invention.

### DETAILED DESCRIPTION

Those skilled in the art will fully understand the terms used herein in the description and the appendant claims to describe the present invention. Nonetheless, unless otherwise provided herein, the following terms are as described immediately below.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification are those which modify an endogenous gene by inserting a “foreign nucleic acid sequence” into the gene locus, e.g., homologous recombination and viral vector gene trapping. The most preferred method of genetic engineering is homologous recombination, in which a foreign nucleic acid sequence is inserted in a targeted manner either alone or in combination with specific nucleotide changes to, or a deletion of, a portion of the endogenous gene sequence.

By “PI3-K” is meant the protein phosphatidylinositol 3-kinase, or sometimes the gene encoding the PI3-K protein. Identical terms are “PI3K” and “PI3-kinase”, and “PI3 kinase”. PI3-K is composed of the p85 regulatory subunit and the p110 enzymatic subunit. Preferably, the mouse p85 subunit of PI3-K has the amino acid sequence provided in Escobedo et al., Cell, 65:75-82 (1991); see also GenBank Acc. No. M60651 and SEQ ID NO: 3.

By a “mutant p85” or “mp85” is meant a gene encoding the p85 subunit that is genetically-modified such that the polypeptide encoded by the mutant gene is unable to bind to and interact properly with the p110 subunit. Thus, because the p85 subunit is mutated, it does not engage fully in its normal activity of binding to and regulating the activity of the p110 subunit. Preferably, the mp85 polypeptide lacks the inter-SH2 domain, although other genetic modifications that disable the ability of mp85 to regulate p110 are contemplated.

By “inter-SH2 domain” is meant that portion of the p85 subunit that is necessary for binding of the p85 subunit to the p110 subunit, and which enables the p85 subunit to properly regulate the p110 subunit. In GenBank Acc. No. M60651, the inter-SH2 domain corresponds approximately to nucleotides 1988-2095; in SEQ ID NO: 3, the inter-SH2 domain corresponds approximately to amino acids 478-513.

By a “genetically-modified, non-human mammal” containing a mutant p85 gene is meant a non-human mammal that is produced, for example, by creating a blastocyst carrying the desired genetic modification and then implanting the blastocyst in a foster mother for in utero development. The genetically-modified blastocyst can be made, in the case of mice, by implanting a genetically-modified embryonic stem (ES) cell into a mouse blastocyst. Alternatively, various species of genetically-modified embryos can be obtained by nuclear transfer. In the case of nuclear transfer, the donor cell is a somatic cell or a pluripotent stem cell, and it is engineered to contain the desired genetic modification. The nucleus of this cell is then transferred into a fertilized or parthenogenetic oocyte that is enucleated, the embryo is reconstituted, and developed into a blastocyst. A genetically-modified blastocyst produced by either of the above methods is then implanted into a foster mother according to standard methods known to those of skill in the art. A “genetically-modified, non-human mammal” includes all progeny of the mammals created by the methods described above, provided that the progeny inherit at least one copy of the introduced genetic modification. It is preferred that all somatic cells and germline cells of the genetically-modified mammal contain the modification. Preferred non-human animals that are genetically-modified include rodents, such as mice and rats, rabbits, guinea pigs, hamsters, pigs, sheep, and ferrets.

By a “genetically-modified animal cell” containing a mutant p85 gene is meant an animal cell, including a human cell, created by genetic engineering to contain a mutant p85 gene, as well as daughter cells that inherit the mutant p85 gene. These cells may be genetically-modified in culture according to any standard method known in the art. The animal cells of the invention may be obtained from primary cell or tissue preparations as well as culture-adapted and/or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germline (EG) cells, and other stem cells, such as progenitor cells and tissue-derived stem cells). The preferred genetically-modified cells are ES cells, more preferably, mouse or rat ES cells, and, most preferably, human ES cells.

By “exogenous gene” is meant a gene that is added to the genome of a cell or organism. An exogenous gene may be derived from the same or different species as the genome to which it is added. An exogenous gene does not replace its counterpart gene in the genome of the cell or animal to which it is added.

By an “ES cell” or an “ES-like cell” is meant a pluripotent stem cell derived from an embryo, from a primordial germ cell, or from a teratocarcinoma, that is capable of indefinite self renewal as well as differentiation into cell types that are representative of all three embryonic germ layers.

By “insulin responsive cell” is meant a cell that expresses the insulin receptor and the Glut4 glucose transporter, such as skeletal muscle cells, cardiac cells, brown and white adipose cells, glomerular cells, renal tubular cells, and certain cells in the brain.

By “insulin responsive cell specific promoter” is meant a promoter that drives the expression of its appended gene primarily only if it is within is an insulin responsive cell. Thus, in a transgenic animal containing a DNA construct wherein the construct has a gene under the control of an insulin responsive cell specific promoter, the gene will primarily only be expressed within insulin responsive cells of the animal, and will not be significantly expressed within other cell types of the animal. Preferred promoters include the Glut4 promoter, the myosin light chain promoter, the creatine kinase promoter, the aP2 promoter, the alpha cardiac myosin heavy chain promoter, the uncoupling protein 3 promoter, the melanin-concentrating hormone promoter, the neuron-specific enolase promoter, the prion promoter, the Thy-1 promoter, the platelet-derived growth factor promoter, the synapsin promoter, and the nestin promoter and enhancer. Most preferred is the Glut4 promoter.

By “reduced” is meant a statistically significant decrease (i.e., p<0.1).

By “increased” is meant a statistically significant increase (i.e., p<0.1).

By “modulates” is meant a statistically significant increase or decrease (including a complete elimination).

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art. Additional guidance with respect to making and using nucleic acids and polypeptides is found in standard textbooks of molecular biology, protein science, and immunology (see, e.g., Davis et al., Basic Methods in Molecular Biology, Elsevier Sciences Publishing, Inc., New York, N.Y. (1986); Hames et al., Nucleic Acid Hybridization, IL Press (1985); Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications cited in this document are herein incorporated by reference.

The living system according to the invention may comprise a transgenic animal. A transgenic animal is one in whose genome a heterologous DNA sequence has been introduced. In particular, the transgenic animal is a transgenic non-human mammal, mammals being generally provided with appropriate signal transduction pathways. For the present purpose, it is generally preferred to employ smaller mammals, e.g., rabbits or rodents such as mice or rats.

For expression of the mutant regulatory p85 subunit of PI3K gene of the invention in transgenic animals, a DNA sequence encoding the mutant regulatory p85 subunit of PI3K is operably linked to additional DNA sequences required for its expression to produce expression units. Such additional sequences include a promoter as indicated herein, as well as sequences providing for termination of transcription and polyadenylation of mRNA. Construction of the expression unit for use in transgenic animals may conveniently be done by inserting a mutant DNA sequence encoding the regulatory p85 subunit of PI3K into a vector containing the additional DNA sequences, although the expression unit may be constructed by essentially any sequence of ligations.

To facilitate detection of mp85 protein, it is preferred to epitope tag the amino terminus so that it can be readily detected by immunoblotting. This is not expected to inhibit the binding of the SH2 domains to phospho-IRS-1 because it has previously been shown that a GST fusion protein that contains both p85 SH2 domains has nanomolar affinity for bifunctional synthetic phosphopeptides derived from IRS-1 containing two YMXM consensus sequences for binding to SH2 domains (Herbst et al., Biochem., 33:9376-81 (1994)). Thus, the amino terminus can tolerate additional protein without affecting binding. Presently preferred epitope tags include myc, influenza hemagglutinin protein (HA), FLAG® epitope, vesicular stomatitis virus glycoprotein (VSV-G), major capsid protein of the T7 phage (T7), and poly-histidine (H₆ or H₁₀), with myc being the presently most preferred epitope tag, partially since it is much smaller than GST. Further information on myc tags and methods of using them can be found at Evans et al., Mol. Cell. Biol., 5:3610 (1985) and Kolodziej et al., Methods Enzymology, 194:508-19 (1991). Of course, epitope tags may be located at any position within the protein, as long as they do not interfere significantly with the activity of the protein, and are not masked by their location within the protein. Thus, epitope tags may be located at the amino-terminus, carboxyl-terminus, or internally within the protein. Typically, terminally located tags are preferred.

The expression unit is then introduced into fertilized ova or early-stage embryos of the selected host species. Introduction of heterologous DNA may be carried out in a number of ways, including microinjection (cf., U.S. Pat. No. 4,873,191), retroviral infection (cf., Jaenisch, Science, 240:1468-74 (1988)), or site-directed integration using embryonic stem cells (reviewed by Bradley et al., Bio/Technology, 10:534-39 (1992)). The ova are then implanted into the oviducts or uteri of pseudopregnant females and allowed to develop to term. Offspring carrying the introduced DNA in their germ line can pass the DNA on to their progeny, allowing the development of transgenic populations.

General procedures for producing transgenic animals are known in the art, cf., for instance, Hogan et al., Manipulating the Mouse Embryo: A Laboratory Manual, Cold Spring Harbor Laboratory, 1986; Simons et al., Bio/Technology, 6:179-83 (1988); Wall et al., Biol. Reprod., 32:645-51 (1985); Buhler et al., Bio/Technology, 8:140-43 (1990); Ebert et al., Bio/Technology, 6:179-83 (1988); Krimpenfort et al., Bio/Technology, 9:844-47 (1991); Wall et al., J. Cell. Biochem., 49:113-20 (1992); U.S. Pat. No. 4,873,191, U.S. Pat. No. 4,873,316; WO 88/100239, WO 90/105188; WO 92/111757; and GB 87/100458. Techniques for introducing heterologous DNA sequences into mammals and their germ cells were originally developed in the mouse. See, e.g. Gordon et al., PNAS, 77:7380-84 (1980), Gordon and Ruddle, Science, 214:1244-46 (1981); Palmiter and Brinster, Cell, 41:343-45 (1985); Brinster et al., PNAS, 82:4438-42 (1985); and Hogan et al. (ibid.). In brief, in the most efficient route used to date in the generation of transgenic mice, several hundred linear molecules of the DNA of interest are injected into one of the pro-nuclei of a fertilized egg according to techniques which have become standard in the art. Injection of DNA into the cytoplasm of a zygote can also be employed. Similar procedures may be employed to produce transgenic individuals of other species.

The present invention provides genetically-modified, non-human mammals that are either heterozygous or homozygous for a genetic modification that introduces a mutant p85 gene. The present invention also provides genetically-modified animal cells, including human cells, that are heterozygous or homozygous for a modification that introduces a mutant p85 gene. The animal cells may be derived by genetically engineering cells in culture, or, in the case of non-human mammalian cells, the cells may be isolated from the above-described genetically-modified, non-human mammals.

Genetically-Modified, Non-human Mammals and Animal Cells

The above-described methods for genetic modification can be used to introduce a mutant p85 gene into virtually any type of somatic or stem cell derived from an animal. Genetically-modified animal cells of the invention include, but are not limited to, mammalian cells, including human cells. These cells may be derived from genetically engineering any animal cell line, such as culture-adapted, tumorigenic, or transformed cell lines, or they may be isolated from a genetically-modified, non-human mammal carrying the desired mutant p85 gene.

Preferred genetically-modified animal cells are ES cells and ES-like cells. These cells are derived from the preimplantation embryos and blastocysts of various species, such as mice (Evans et al., Nature, 129:154-56 (1981); Martin, PNAS, 78:7634-38, (1981)), pigs and sheep (Notanianni et al., J. Reprod. Fert., Suppl. 43:255-60, (1991); Campbell et al., Nature, 380:64-68 (1996)), and primates, including humans (Thomson et al., U.S. Pat. No. 5,843,780; Thomson et al., Science, 282:1145-47 (1995); and Thomson et al., PNAS, 92:7844-48 (1995)).

These types of cells are pluripotent. That is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, hepatocytes, pancreatic, β-cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol., 7:862-69, (1995); Li et al., Curr. Biol., 8:971 (1998); Klug et al., J. Clin. Invest., 98:216-24 (1996); Lieschke et al., Exp. Hematol., 23:328-34 (1995); Yamane et al., Blood, 90:3516-23 (1997); and Hirashima et al., Blood, 93:1253-63 (1999).

Genetically-modified murine ES cells may be used to generate genetically-modified mice. Embryonic stem cells are manipulated according to published procedures (Robertson, Teratocarcinomas and Embryonic Stem Cells: A Practical Approach, Ed. E. J. Robertson, Oxford: IRL Press, pp. 71-112 (1987); Zjilstra et al., Nature, 342:435-38 (1989); and Schwartzberg et al., Science, 246:799-803 (1989)). The particular embryonic stem cell line employed is not critical; exemplary murine ES cell lines include AB-1 (McMahon and Bradley, Cell, 62:1073-85 (1990)), E14 (Hooper et al., Nature, 326:292-95 (1987)), D3 (Doetschman et al., J. Embryol. Exp. Morph., 87:27-45 (1985)), CCE (Robertson et al, Nature, 323:445-48 (1986)), RW4 (Genome Systems, St. Louis, Mo.), and DBA/1lacJ (Roach et al., Exp. Cell Res., 221:520-25 (1995)).

Following confirmation that the ES cells contain the desired mutant p85 gene, these ES cells are then injected into suitable blastocyst hosts for generation of chimeric mice according to methods known in the art (Capecchi, Trends Genet., 5:70 (1989)). The particular mouse blastocysts employed in the present invention are not critical. Examples of such blastocysts include those derived from C57BL/KsJ mice, C57BL6 mice, C57BL6 Albino mice, Swiss outbred mice, CFLP mice, and MFI mice. Alternatively, ES cells may be sandwiched between tetraploid embryos in aggregation wells (Nagy et al., PNAS, 90:8424-28 (1993)).

The blastocysts containing the genetically-modified ES cells are then implanted in pseudopregnant female mice and allowed to develop in utero (Hogan et al., Manipulating the Mouse Embryo: A Laboratory Manual, Cold Spring Harbor Laboratory, 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the mutant p85 gene. Such offspring contain some cells that are derived from the genetically-modified donor ES cell as well as other cells from the original blastocyst. Offspring may be screened initially for mosaic coat color where a coat color selection strategy has been employed to distinguish cells derived from the donor ES cell versus the other cells of the blastocyst. Alternatively, DNA from tail tissue of the offspring can be used to identify mice containing the genetically-modified cells.

The mating of chimeric mice that contain the mp85 gene in germ line cells produces progeny that possess the mp85 gene in all germ line cells and somatic cells. Mice that are heterozygous for the mp85 gene can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method is not limited to making mice; it can be employed to make other genetically-modified, non-human mammals, for example, sheep (McCreath et al., Nature, 29:1066-69 (2000); Campbell et al., Nature, 389:64-66 (1996); and Schnieke et al., Science, 278:2130-33 (1997)) and calves (Cibelli et al., Science, 280:1256-58, (1998)). Briefly, somatic cells (e.g., fibroblasts) or pluripotent stem cells (e.g., ES-like cells) are selected as nuclear donors and are genetically-modified to contain a mutant p85 gene. Nuclei from donor cells which have the appropriate mp85 gene are then transferred to fertilized or parthenogenetic oocytes that are enucleated (Campbell et al., Nature, 380:64 (1996); Wilmut et al., Nature, 385:810 (1997)). Embryos are reconstructed, cultured to develop into the morula/blastocyst stage, and transferred into foster mothers for in utero full term development.

The present invention also encompasses the progeny of the genetically-modified, non-human mammals and genetically-modified animal cells. While the progeny are heterozygous or homozygous for the genetic modification, they may not be genetically identical to the parent non-human mammals and animal cells due to mutations or environmental influences that may occur in succeeding generations.

The transgenic animals and cell lines produced according to the present invention are useful in methods of screening for new drugs. For example, the p85DN transgenic mice (see Example 2) can be screened for drugs that reduce the body fat composition of these mice, making them more like their wild-type counterparts. For example, changes in body fat composition of animals can be measured by DEXA analysis (see Example 3), CAT scans (high resolution x-ray microcomputed tomography, or microCT, preferably the MicroCT devices and methods of ImTek, Inc., Oak Ridge, Tenn.), correlation to plasma leptin levels, as well as by direct measurement. Likewise, certain biological and enzymatic markers can be followed in the transgenic animals and cells produced according to the invention, and the effect of test compounds on these markers measured. Markers include plasma leptin levels, beta-hydroxybutyrate, triglyceride, free fatty acids, cholesterol, lactate, glucose, insulin, and corticosterone. Examples of cell assays include, but are not limited to, assessing lipogenesis, lipolysis and leptin secretion in adipocytes. Differentiation of pre-adipocytes into adipocytes can be assessed by quantitating intracellular triglyceride accumulation or measuring the induction of adipocyte-specific genes such as resistin, aP2, and ACRP 30. Differentiation of myoblast cells into myocytes can be assessed by measuring the induction of muscle-specific genes such as myogenin.

Examples of agents that are screened include, but are not limited to, nucleic acids (e.g., DNA, RNA, and antisense RNA), carbohydrates, lipids, proteins, antibodies, peptides, peptidomimetics, small molecules, and other agents. Agents can be selected individually for testing or as part of a library. These libraries are obtained using any of the numerous approaches in combinatorial library methods known in the art, and include: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (e.g., Lam, Anticancer Drug Des., 12:145 (1997); U.S. Pat. No. 5,738,996; and U.S. Pat. No. 5,807,683).

Examples of methods for the synthesis of molecular libraries can be found in the art, for example, in DeWitt et al., PNAS, 90:6909 (1993), Erb et al., PNAS, 91:11422 (1994), Zuckermann et al., J. Med. Chem., 37:2678 (1994), Cho et al., Science, 261:1303 (1993), Carrell et al., Angew. Chem. Int. Ed. Engl., 33:2059 (1994), Carell et al., Angew. Chem. Int. Ed. Engl., 33:2061 (1994), and Gallop et al., J. Med. Chem., 37:1233 (1994).

Individual agents or libraries of agents may be presented in solution (e.g., Houghten, Bio/Techniques, 13:412-421 (1992)), or on beads (Lam, Nature, 354:82-84 (1991)), chips (Fodor, Nature, 364:555-556 (1993)), bacteria (U.S. Pat. No. 5,223,409), spores (U.S. Pat. Nos. 5,571,698; 5,403,484; and 5,223,409), plasmids (Cull et al., PNAS, 89:1865-69 (1992)) or phage (Scott and Smith, Science, 249:386-90 (1990); Devlin, Science, 249:404-06 (1990); Cwirla et al., PNAS, 87:6378-82 (1990); and Felici, J. Mol. Biol., 222:301-10 (1991)).

### EXAMPLES

### Example 1

### Preparation of the mp85 DNA Construct

mp85 encoding cDNA was produced by two PCR fragments from mouse cDNA using primers Xba I-p85.1B (AGCTCTAGAGTGGCAGGGCACT; SEQ ID NO: 4) and BgI II-p85.2B (TTTAGATCTGATTTCCTGGGAAG; SEQ ID NO: 5) for the 5′ end, and BgI II-p85.3B (AAGAGATCTCGCGAAGGCAACG; SEQ ID NO: 6) and BamHI-p85.4M (CAGCGCGGATCCGCCTCTGTTGTGC; SEQ ID NO: 7) for the 3′ end. After appropriate digests (Xba I/BgI II for 5′ end and BgI II/Bam HI for 3′ end), these two PCR fragments were ligated first to each other and then into the Xba I/Bam HI site of pBluescript myc. pBluescript myc is a plasmid created by digesting a c-myc epitope cassette (SEQ ID NO: 8) with Sma I and ligating the resulting fragment into the Sma I site of pBluescript II SK (Stratagene, La Jolla, Calif.). This created a nucleotide sequence encoding a mutant p85 protein having a 36 amino acid deletion of the p110 DNA binding domain encompassing amino acids 478-513 of the p85 gene (see SEQ ID NO: 3), resulting in a mp85 protein having the sequence shown in SEQ ID NO: 1.

The Glut 4 promoter was cloned by PCR using Xba I linked primers at 445bp-473bp at 5′ end (GAGTCTAGATCACTCTGTCG; SEQ ID NO: 9) and 2511bp-2530bp at 3′ end (GACTCTAGAGTCCGATGG; SEQ ID NO: 10) of plasmid pG4CHIII obtained from Dr. Jeffrey Pessin from the University of Iowa. The full amplified sequence corresponds to bp122-bp2185 of the human glut4 gene 5′ flanking region sequence seen in GenBank Acc. No. M61126 (see SEQ ID NO: 11 for the full amplified sequence). The PCR fragment was digested with Xba I and cloned into the Xba I site of pBluescript myc containing the mp85 gene. The entire hGH gene was cut out as a CIa I/Hind III fragment from plasmid pZAP-N (see Gladue et al., Clin. Exp. Immunol., 110:397-402 (1997); Seeburg, DNA, 1 (3):239-49 (1982); and GenBank Acc. No. M13438), polished to blunt ends using T4 DNA polymerase to fill in 5′ or 3′ overhangs, and ligated to Eco RI linkers containing STOP codons in three reading frames (CCGGAATTCTGATAGTAA; SEQ ID NO: 12). After Eco RI digestion, the resulting fragment was cloned into the Eco RI site of pBluescript myc containing the mp85 gene plus the Glut 4 promoter (supra). The entire transgene was separated from backbone plasmid for microinjection. Before microinjection, the transgene was purified by Not I/Xho I digest, gel isolated, purified on an Elutip column (Schleicher and Schuell catalog #27360), concentrated by ethanol precipitation, and redissolved in TE (Tris 1 mM, EDTA 0.1 mM). The resulting DNA construct has the nucleotide sequence shown in SEQ ID NO: 2.

### Example 2

### Production of p85DN Transgenic Mice

Pronuclear transgenic mice were produced by established protocols (Hogan et al., in Manipulating the Mouse Embryo, A Laboratory Manual (Cold Spring Harbor, 1986); DePamphilis et al., Biotechniques, 6:662-88 (1998)) using embryos obtained from the inbred FVB strain of mice. The 6.6 kb (Not I-Xho I fragment) mutant mouse p85 transgene construct of Example 1 was microinjected at about 2.0 μg/ml into pronuclei of embryos derived from matings of FVB males and superovulated females (Taconic, Germantown, N.Y.). Four hundred twenty seven injected embryos were transferred to pseudopregnant CD-1 females (Charles Rivers Laboratories, Wilmington, Mass.). Of 68 offspring, 12 founder transgenic mice were identified by the polymerase chain reaction (PCR) of total genomic DNA. Independent lines were established by backcrossing founder mice with FVB mates.

### Example 3

### DEXA Analysis of Body Fat Composition

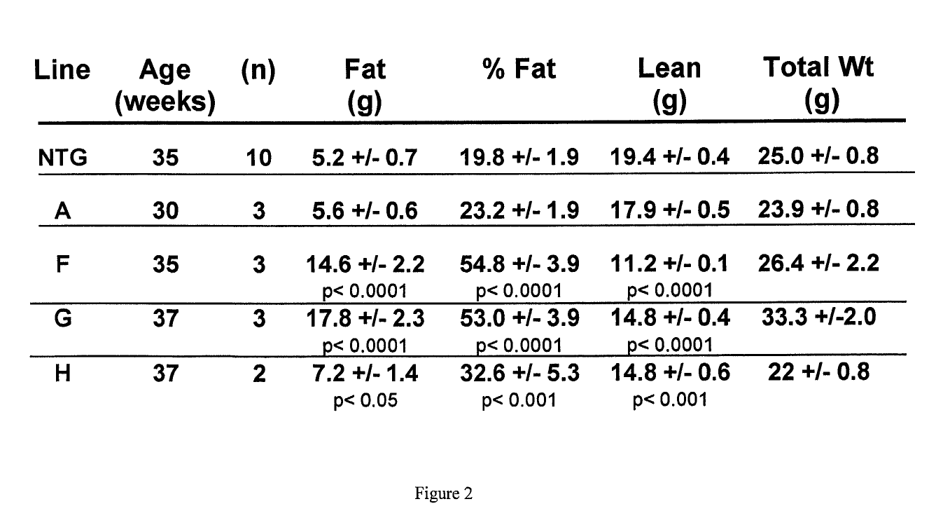

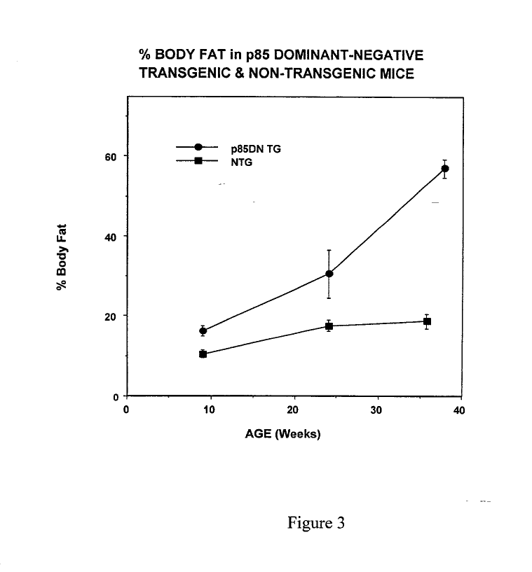

p85DN transgenic mice and their nontransgenic littermates under ketamine/xylazine anesthesia underwent dual-energy X-ray absorptiometry (DEXA, QDR-1000/W, Hologic Inc., Waltham, Mass.) equipped with a Whole Body Scan software for lean and fat body mass determination. Total weight, lean and fat body mass, and percent lean and fat body mass were determined (Ke et al., Endocrinology, 139:2068-76 (1998). In FIG. 2, both wild-type mice and mice from p85DN transgenic lines A, F, G, and H are shown, and measurements were taken when the mice were between 30 and 37 weeks of age. In FIG. 3, DEXA analysis was performed on mice from the non-transgenic group as well as p85DN transgenic mice from line G at various age points to determine when the body fat composition variations occurred.

Transgenic mice carrying the p85DN gene exhibited significantly higher body fat and significantly lower lean muscle mass than their wild-type counterparts. See FIGS. 2 and 3.

### Example 4

### Oral Glucose Tolerance Test of p85DN Transgenic Mice

Non-anesthetized p85DN transgenic mice and their nontransgenic littermates were fasted overnight and then bled via the orbital sinus (0.025 ml) immediately prior to administration of an oral glucose load (2 g glucose per kg of body weight) by gavage using a syringe equipped with a murine oral feeding needle (20 gauge; Popper & Sons, Inc., New Hyde Park, N.Y.). The 0.025 ml blood sample was added to 0.1 ml of 0.025% heparinized-saline in Denville Scientific microtubes (South Plainfield, N.J.). The tubes were spun at the highest setting in a Beckman Microfuge 12 (Beckman Coulter, Fullerton, Calif.) for 2 minutes. Plasma was collected for plasma glucose determination using the VP Super System Autoanalyzer (Abbott Laboratories, North Chicago, Ill.). Mice were subsequently bled after 30, 75, and 120 min and plasma glucose determined in the same manner.

Transgenic mice carrying the p85DN gene exhibited the same ability to dispose of an oral glucose load as did their wild-type counterparts.

### Example 5

### Insulin Tolerance Test of p85DN Transgenic Mice

Non-anesthetized p85DN transgenic mice and their nontransgenic littermates in the fed state were bled via the orbital sinus (0.025 ml) immediately prior to administration of a subcutaneous insulin injection (2 Units/kg body weight). After injection, mice were bled at 30, 75, and 120 minutes and plasma glucose was determined as described in Example 4.

Transgenic mice carrying the p85DN gene exhibited the same response to a subcutaneous insulin injection as did their wild-type counterparts.

### Example 6

### Glycogen Content Analysis of p85DN Transgenic Mice Tissues

p85DN transgenic mice and nontransgenic mice in the fed or overnight-fasted (18 h) state were killed by decapitation and then mixed hindlimb skeletal muscle, heart, and liver samples were rapidly isolated and then clamp frozen in liquid N₂ cooled aluminum tongs; tissue glycogen levels were then determined by the method of Hassid and Abraham (Methods Enzym., 3:34-35 (1959)). Frozen tissue (approximately 150 mg) was cut into 20-30 mg blocks taking care to prevent the tissue from thawing. The frozen tissue was placed in test tubes and dissolved in 0.3 ml of 30% KOH (w/v) by immersion in boiling water for 1 hr. Glycogen was precipitated by the addition of 0.2 ml of 2% (w/v) Na₂SO₄ and 2 ml of ice-cold absolute ethanol, and the samples were stored overnight at −20° C. The next day samples were centrifuged at 3500 rpm for 10 minutes and the supernatant was discarded. The resulting pellet was washed once with 2 ml of ice-cold 66% ethanol. Glycogen was converted into monosaccharides by boiling the pellet in 1 ml of 3.79 M H₂SO₄ for 3 hrs. Following this, 0.1 ml of 0.33 M MOPS was added to each tube and the solution was neutralized with 10 M KOH to pH 7. The final volume of the solution was measured and glucose concentration was determined by using a glucose determination kit (Sigma, St. Louis, Mo.).

Liver, skeletal muscle, and cardiac glycogen content were the same in transgenic mice carrying the p85DN gene and their wild-type counterparts.

### Example 7

### 2-Deoxyqlucose Uptake in Isolated Soleus Muscle of p85DN Transgenic Mice

Non-fasted male p85DN transgenic mice and their nontransgenic littermates weighing approximately 30 g were anesthetized via an i.p. injection of pentobarbital sodium (6.5 mg/100 g body weight). Soleus muscles (approximately 12 mg) were then isolated and incubated individually in a manner similar to that described previously (Etgen et al., Am. J. Physiol., 271:E294-E301 (1996)). Briefly, soleus muscles were preincubated for 90 min at 29° C. in 1.8 ml of continuously gassed (95% O₂-5% CO₂) Krebs-Henseleit bicarbonate buffer (KHB) containing 0.1% bovine serum albumin (BSA), 8 mM glucose, and 32 mM mannitol. After the preincubation phase, muscles were washed in KHB (1.8 ml at 29° C.) containing 0.1% BSA and 40 mM mannitol. Muscles were then transferred to fresh KHB and glucose transport was measured over 20 min at 29° C. in the presence of 1 mM 2-deoxy-D-glucose (2.25 mCi/mmol 2-[3H(G)]-deoxy-D-glucose), 39 mM mannitol (10 μCi/mmol [U-¹⁴C]-D-mannitol), and 2 mM sodium pyruvate. Following the final incubation phase, muscles were blotted on gauze and then clamp frozen in liquid N₂ cooled aluminum tongs. Frozen muscle was trimmed of any tendon, weighed, and digested in 0.5 ml of 1N KOH heated to 60° C. for 1 hour. The digest was then cooled on ice and 0.5 ml of 1N HCl was added to neutralize it. A 0.3 ml aliquot was then added to 5 ml of Beckman Coulter Ready-Safe scintillation cocktail (Fullerton, Calif.) and counted on an LKB-Wallac 1219 Rack Beta scintillation counter (Perkin Elmer Wallac, Gaithersburg, Md.). Glucose transport activity, expressed in μmol/g/20 min, was calculated from the intracellular 2-[3H]-deoxy-D-glucose accumulation using [U-¹⁴C]mannitol as the extracellular marker. When the effects of insulin were examined, the hormone was present during preincubation, wash, and incubation phases.

2-Deoxyglucose uptake in isolated soleus muscle under both basal or insulin-stimulated conditions was the same in transgenic mice carrying the p85DN gene as in their wild-type counterparts.

### Example 8

### Correlation of Fat Pad Weight and Plasma Leptin Levels

Following body composition analysis by DEXA as described above, p85DN transgenic mice and nontransgenic littermates were killed by decapitation and about 1 ml of blood was collected in Becton-Dickinson Microtainer® brand plasma separator tubes (Franklin Lakes, N.J.) with lithium heparin. The tubes were spun in a Beckman Microfuge 12 at the maximum setting for five minutes. Plasma was collected in 1.5 ml Eppendorf tubes and snap frozen in liquid nitrogen. Plasma samples were stored at −80° C. until analyzed for leptin levels. Plasma leptin was quantitated using an Enzyme Immuno Assay (Assay Designs Rat Leptin Correlate Kit, Ann Arbor, Mich.). Gonadal fat pads were rapidly removed and then clamp frozen in liquid N₂ cooled aluminum tongs. Frozen fat pads were weighed, wrapped in aluminum foil and stored at −80° C. for further analysis.

Plasma leptin levels were significantly correlated (R²=0.949) in a positive manner with gonadal fat pad weight in transgenic mice carrying the p85DN gene and their wild-type counterparts.

1. A genetically-modified, non-human mammal having an altered body fat composition, wherein said mammal comprises an exogenous mutant p85 PI3-K gene, wherein the expression of said gene is driven by an insulin responsive cell specific promoter.

2. The mammal of claim 1, wherein said mutant p85 PI3-K gene encodes a mutant p85 PI3-K protein lacking the inter-SH2 domain.

3. The mammal of claim 2, wherein said mutant p85 PI3-K protein has the amino acid sequence of SEQ ID NO: 1.

4. The mammal of claim 1, wherein said mutant p85 PI3-K gene encodes a p85 PI3-K protein whose inter-SH2 domain is unable to functionally interact with the p110 subunit of PI3-K.

5. The mammal of claim 1, wherein said insulin responsive cell specific promoter is selected from the group consisting of the GLUT4 promoter, the myosin light chain promoter, the creatine kinase promoter, the aP2 promoter, the alpha cardiac myosin heavy chain promoter, the uncoupling protein 3 promoter, the melanin-concentrating hormone promoter, the neuron-specific enolase promoter, the prion promoter, the Thy-1 promoter, the platelet-derived growth factor promoter, the synapsin promoter, and the nestin promoter and enhancer.

6. The mammal of claim 5, wherein said insulin responsive cell specific promoter is the GLUT4 promoter.

7. The mammal of claim 1, wherein said mammal is a rodent.

8. The rodent of claim 7, wherein said rodent is a mouse.

9. A genetically-modified mouse having an altered body fat composition, wherein said mouse comprises an exogenous mutant p85 PI3-K gene having the amino acid sequence of SEQ ID NO: 1, wherein the expression of said gene is driven by the Glut4 promoter.

10. A genetically-modified animal cell, wherein said cell comprises an exogenous mutant p85 PI3-K gene, wherein the expression of said gene is driven by an insulin responsive cell specific promoter.

11. The animal cell of claim 10, wherein said mutant p85 PI3-K gene encodes a mutant p85 PI3-K protein lacking the inter-SH2 domain.

12. The animal cell of claim 11, wherein said mutant p85 PI3-K protein has the amino acid sequence of SEQ ID NO: 1.

13. The animal cell of claim 10, wherein said insulin responsive cell specific promoter is selected from the group consisting of the GLUT4 promoter, the myosin light chain promoter, the creatine kinase promoter, the aP2 promoter, the alpha cardiac myosin heavy chain promoter, the uncoupling protein 3 promoter, the melanin-concentrating hormone promoter, the neuron-specific enolase promoter, the prion promoter, the Thy-1 promoter, the platelet-derived growth factor promoter, the synapsin promoter, and the nestin promoter and enhancer.

14. The animal cell of claim 10, wherein said insulin responsive cell specific promoter is the GLUT4 promoter.

15. The animal cell of claim 10, wherein said cell is an embryonic stem (ES) cell or an ES-like cell.

16. The animal cell of claim 10, wherein said cell is human.

17. The animal cell of claim 10, wherein said cell is murine.

18. A genetically-modified mouse cell, wherein said cell comprises an exogenous mutant p85 PI3-K gene having the amino acid sequence of SEQ ID NO: 1, wherein the expression of said gene is driven by the Glut4 promoter.

19. A method of identifying an agent that modulates the body fat composition of a mammal, said method comprising the steps of
comparing the body fat compositions of a test animal to a control animal, wherein said agent has been administered to said test animal, further wherein each of said test and control animals is a genetically-modified, non-human mammal having an increased body fat composition, wherein said mammal comprises an exogenous mutant p85 PI3-K gene, wherein the expression of said gene is driven by an insulin responsive cell specific promoter; and
identifying as a modulating agent the agent that has a statistically significant effect on the body fat composition of the test animal.

20. The method of claim 19 wherein the body fat composition of said test animal and said control animal is measured using DEXA analysis, CAT scans, correlation to plasma leptin levels, or by direct measurement.

21. The method of claim 19, wherein said insulin responsive cell specific promoter is the GLUT4 promoter.

22. The method of claim 19, wherein said mammal is a mouse.

23. A DNA expression construct for expressing a mutant p85 PI3-K gene, said construct comprising:
a mutant p85 PI3-K gene, wherein said gene encodes a p85 PI3-K protein whose inter-SH2 domain is unable to functionally interact with the p110 subunit of PI3-K; and
an insulin responsive cell specific promoter that controls the expression of said gene.

24. The DNA expression construct of claim 23, wherein gene encodes a mutant p85 PI3-K protein lacking the inter-SH2 domain.

25. The DNA expression construct of claim 24, wherein said mutant p85 PI3-K protein has the amino acid sequence of SEQ ID NO:1.

26. The DNA expression construct of claim 23, wherein said insulin responsive cell specific promoter is selected from the group consisting of the GLUT4 promoter, the myosin light chain promoter, the creatine kinase promoter, the aP2 promoter, the alpha cardiac myosin heavy chain promoter, the uncoupling protein 3 promoter, the melanin-concentrating hormone promoter, the neuron-specific enolase promoter, the prion promoter, the Thy-1 promoter, the platelet-derived growth factor promoter, the synapsin promoter, and the nestin promoter and enhancer.

27. The DNA expression construct of claim 26, wherein said insulin responsive cell specific promoter is the GLUT4 promoter.

28. A DNA expression construct for expressing a mutant p85 PI3-K gene, said construct comprising a mutant p85 PI3-K gene, wherein said gene encodes a mutant p85 PI3-K protein having the amino acid sequence of SEQ ID NO: 1, and a Glut4 promoter that controls the expression of said gene.


SEQUENCE LISTING (12 sequences)

SEQ ID NO: 1 - protein, 688 aa, mus musculus
MSAEGYQYRALYDYKKEREEDIDLHLGDILTVNKGSLVALGFSDGPEARPEDIGWLNGYNETTGERGDFPGTYVEYIGRKRISPPTPKPRPPRPLPVAPGSSKTEADTEQQALPLPDLAEQFAPPDVAPPLLIKLLEAIEKKGLECSTLYRTQSSSNPAELRQLLDCDAASVDLEMIDVHVLADAFKRYLADLPNPVIPVAVYNEMMSLAQELQSPEDCIQLLKKLIRLPNIPHQCWLTLQYLLKHFFKLSQASSKNLLNARVLSEIFSPVLFRFPAASSDNTEHLIKAIEILISTEWNERQPAPALPPKPPKPTTVANNSMNNNMSLQDAEWYWGDISREEVNEKLRDTADGTFLVRDASTKMHGDYTLTLRKGGNNKLIKIFHRDGKYGFSDPLTFNSVVELINHYRNESLAQYNPKLDVKLLYPVSKYQQDQVVKEDNIEAVGKKLHEYNTQFQEKSREYDRLYEEYTRTSQEIREGNEKEIQRIMHNHDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 2 - DNA, 6591 bp, Artificial Sequence
  DNA construct expressing mp85
tggagctccaccgcggtggcggccgctctagatgcatgctcgagcggccgccagtgtgatggatatctgcagaattcggcttgagtctagatcactctgtcgccaggctggagtgcaagggcacgatcttggctcactacaacctccacctcctgggttcaagccattttcctgcctcagcctcccgagtagctgggattacaggtgtgcataaccacgcccggctaatttttgtatctttagcagacatggggtttctctatgttggccaggctggtttcaaactcctgacctcagtcgatccacctgccttggcctcctaaagtgctgggattacaggcatgagccaccaggccgggccggcattccagatttttcaggggattcgtgcagcaaaggaatcaagaagggatgtaaaggcacagtgtgttctgggtacaataaggacttaggcattgcccagaacaggaggcgaaggagatagaaggagaggcaggagagataggtaaggccagagatcggataagagaggcaggaggttttgttcactctgaaaagggatttgaacttggcaattggggcaacagagacagtgacttcttgcttgagagatgagattggaccttcgaaaattgttctctgccctcgtcataaaggaaataagaggagcacgaagaccagtgagggtgatggtgatctggactgaagtggcagccgccacggagaatatcggatgaatgtgagagagttttggaggtcaaagcaccaatgttggaaactaactggataaacgaggagagcggcgcaggacaggaggaatcgagcctgacttctaccataggggtgactgggcgggtaattcattgaaataaggaagttaggaggaggagcaggtttggacatgctgatcactagagctgccacatccgggcggtaacgaacacctggatctgcagctccagagaagggcctgggtcagatgtcactgaagccctatggtggcggaaaggcgagaaatagtgggttgagattccaagtgcaatccactgcggctcctcgctcgccctccaggtggcagcacaaccctgcgcttccgaagcccgttttctgagccagacactctccacgctctgggtatttcggcttctctctccccacacgccgaccctaggtcgcgcactttctgcctggcagaatttggccgaggatccaaacccggagcagcctccagagagcgtgtcgttcacgcggccagcatatgctcagagacctcagaggctcagagacctcagggctggtggtgtggtcggttgtgaccacttgtccctcggaccggctccaggaaccaacctggggaatgtgtgtaggggaagggcgggatagacagtgcccggagcagggaggcgctgaaagacaggaccaagcagcccggccaccagacccgttgtgggaacggaatttcctggcccccagggccacactcgcgtgggaagcatgtcgcggaccctttaaggcgtcatctccctgtctctccgcccccgcctgggacaggccgggacgcccgggacctgacatttggaggctcccaacgtgggagctaaaaatagcagccccgggttactttggggcattgctcctctcccaacccgcgcgccggctcgcgagccgtctcaggccgctggagtttccccggggcaagtacacctggcccgtcctctcctctcagaccccactgtccagacccgcagagtttaagatgcttctgcagcccgggatcctagctggtgggcggagtcctaacacgtgggtgggcggggccttttgttccagggactcttttctcaaaacttcccagtcggaggctggcgggaacccgagaggcgtgtctcgccagccacgcggaggggcgtggcctcattggcccgccccaccaactccagccaaactctaaaccccaggcggagggggcgtggccttctggggtgtgcgggctcctggccaatgggtgctgtgaagggcgtggcccgcgggggcaggagcgaggtggcgggggcttctcgcgtcttttcccccagccccgctccacaagatccgcgggagccccactgctctccggatccttggcttgtggctgtgggtcccatcggactctagagtggcagggcactagagctgcagacatgagtgcagagggctaccagtacagagcactgtacgactacaagaaggagcgagaggaagacattgacctacacctgggggacatactgactgtgaataaaggctccttagtggcacttggattcagtgatggccaggaagcccggcctgaagatattggctggttaaatggctacaatgaaaccactggggagaggggagactttccaggaacttacgttgaatacattggaaggaaaagaatttcaccccctactcccaagcctcggccccctcgaccgcttcctgttgctccgggttcttcaaaaactgaagctgacacggagcagcaagcgttgccccttcctgacctggccgagcagtttgcccctcctgatgttgccccgcctctccttataaagctcctggaagccattgagaagaaaggactggaatgttcgactctatacagaacacaaagctccagcaaccctgcagaattacgacagcttcttgattgtgatgccgcgtcagtggacttggagatgatcgacgtacacgtcttagcagatgctttcaaacgctatctcgccgacttaccaaatcctgtcattctgtagctgtttacaatgagatgatgtctttagcccaagaactacagagccctgaagactgcatccagctgttgaagaagctcattagattgcctaatatacctcatcagtgttggcttacgcttcagtatttgctcaagcattttttcaagctctctcaagcctccagcaaaaaccttttgaatgcaagagtcctctctgagattttcagccccgtgcttttcagatttccagccgccagctctgataatactgaacacctcataaaagcgatagagattttaatctcaacggaatggaatgagagacagccagcaccagcactgccccccaaaccacccaagcccactactgtagccaacaacagcatgaacaacaatatgtccttgcaggatgctgaatggtactggggagacatctcaagggaagaagtgaatgaaaaactccgagacactgctgatgggacctttttggtacgagacgcatctactaaaatgcacggcgattacactcttacactaaggaaaggaggaaataacaaattaatcaaaatctttcaccgtgatggaaaatatggcttctctgatccattaaccttcaactctgtggttgagttaataaaccactaccggaatgagtctttagctcagtacaaccccaagctggatgtgaagttgctctacccagtgtccaaataccagcaggatcaagttgtcaaagaagataatattgaagctgtagggaaaaaattacatgaatataatactcaatttcaagaaaaaagtcgggaatatgatagattatatgaggagtacacccgtacttcccaggaaatcagatctcgcgaaggcaacgagaaagaaattcaaaggattatgcataaccatgataagctgaagtcgcgtatcagtgagatcattgacagtaggaggaggttggaagaagacttgaagaagcaggcagctgagtaccgagagatcgacaaacgcatgaacagtattaagccggacctcatccagttgagaaagacaagagaccaatacttgatgtggctgacgcagaaaggtgtgcggcagaagaagctgaacgagtggctggggaatgaaaataccgaagatcaatactccctggtagaagatgatgaggatttgccccaccatgacgagaagacgtggaatgtcgggagcagcaaccgaaacaaagcggagaacctattgcgagggaagcgagacggcactttccttgtccgggagagcagtaagcagggctgctatgcctgctccgtagtggtagacggcgaagtcaagcattgcgtcattaacaagactgccaccggctatggctttgccgagccctacaacctgtacagctccctgaaggagctggtgctacattatcaacacacctccctcgtgcagcacaatgactccctcaatgtcacactagcatacccagtatatgcacaacagaggcggatccccccccggggaggcctgtcgcgatccggaccggaacaaaagcttatttctgaagaagacttggctagcagtactcgccggcccccgggggctgcaggaattccgatgatcccaaggcccaactccccgaaccactcagggtcctgtggacagctcacctagcggcaatggctacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgtaagctcttggggaatgggtgcgcatcaggggtggcaggaaggggtgactttcccccgctgggaaataagaggaggagactaaggagctcagggtttttcccgaagcgaaaatgcaggcagatgagcacacgctgagtgaggttcccagaaaagtaacaatgggagctggtctccagcgtagaccttggtgggcggtccttctcctaggaagaagcctatatcccaaaggaacagaagtattcattcctgcagaacccccagacctccctctgtttctcagagtctattccgacaccctccaacagggaggaaacacaacagaaatccgtgagtggatgccttctccccaggcggggatgggggagacctgtagtcagagcccccgggcagcacagccaatgcccgtccttcccctgcagaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttcctttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgatatcaagcttatcgataccgtcgacctcgagggggggcccggtacccaattcgccctatagtgagtcgtat

SEQ ID NO: 3 - protein, 724 aa, mus musculus
MSAEGYQYRALYDYKKEREEDIDLHLGDILTVNKGSLVALGFSDGPEARPEDIGWLNGYNETTGERGDFPGTYVEYIGRKRISPPTPKPRPPRPLPVAPGSSKTEADTEQQALPLPDLAEQFAPPDVAPPLLIKLLEAIEKKGLECSTLYRTQSSSNPAELRQLLDCDAASVDLEMIDVHVLADAFKRYLADLPNPVIPVAVYNEMMSLAQELQSPEDCIQLLKKLIRLPNIPHQCWLTLQYLLKHFFKLSQASSKNLLNARVLSEIFSPVLFRFPAASSDNTEHLIKAIEILISTEWNERQPAPALPPKPPKPTTVANNSMNNNMSLQDAEWYWGDISREEVNEKLRDTADGTFLVRDASTKMHGDYTLTLRKGGNNKLIKIFHRDGKYGFSDPLTFNSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIGKFKREGNEKEIQRIMHNHDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 4 - DNA, 22 bp, Artificial Sequence
  xba I-p85.1B primer
agctctagagtggcagggcact

SEQ ID NO: 5 - DNA, 23 bp, Artificial Sequence
  Bg1 II-p85.2B primer
tttagatctgatttcctgggaag

SEQ ID NO: 6 - DNA, 22 bp, Artificial Sequence
  primer
aagagatctcgcgaaggcaacg

SEQ ID NO: 7 - DNA, 25 bp, Artificial Sequence
  BamHI-p85.4M primer
cagcgcggatccgcctctgttgtgc

SEQ ID NO: 8 - DNA, 85 bp, Artificial Sequence
  c-myc epitope cassette
cccggggaggcctgtcgcgatccggaccggaacaaaagcttatttctgaagaagacttggctagcagtactcgccggcccccggg

SEQ ID NO: 9 - DNA, 20 bp, Artificial Sequence
  primer
gagtctagatcactctgtcg

SEQ ID NO: 10 - DNA, 18 bp, Artificial Sequence
  primer
gactctagagtccgatgg

SEQ ID NO: 11 - DNA, 2064 bp, Artificial Sequence
  bp122-bp2185 of the human glut4 gene 5′ flanking region sequence seen in GenBank Acc. No. M61126
gagacggagtcactctgtcgccaggctggagtgcaagggcacgatcttggctcactacaacctccacctcctgggttcaagccattttcctgcctcagcctcccgagtagctgggattacaggtgtgcataaccacgcccggctaatttttgtatctttagcagacatggggtttctctatgttggccaggctggtttcaaactcctgacctcagtcgatccacctgccttggcctcctaaagtgctgggattacaggcatgagccaccaggccgggccggcattccagatttttcaggggattagtgcagcaaaggaatcaagaagggatgtaaaggcacagtgtgttctgggtacaataaggacttaggcattgcccagagcaggaggcgaaggagatagaaggagaggcaggagagataggtaaggccagagatcggataagagaggcaggaggttttgttcactctgaaaagggatttgaacttggcaattggggcaacagagacagtgacttcttgcttgagagatgagattggaccttcgaaaattgttctctgccctcgtcataaaggaaataagaggagcacgaagaccagtgagggtgatggtgatctggactgaagtggcagccgccacggagaatatcggatgaatgtgagagagttttggaggtcaaagcaccaatgttggaaactaactggataaacgaggagagcggcgcaggacaggaggaatcgagcctgacttctaccataggggtgactgggcgggtaattcattgaaataaggaagttaggaggaggagcaggtttggacatgctgatcactagagctgccacatccgggcggtaacgaacacctggatctgcagctccagagaagggcctgggtcagatgtcactgaagccctatggtggcggaaaggcgagaaatagtgggttgagattccaagtgcaatccactgcggctcctcgctcgccctccaggtggcagcacaaccctgcgcttccgaagcccgttttctgagccagacactctccacgctctgggtatttcggcttctctctccccacacgccgaccctaggtcgcgcactttctgcctggcagaatttggccgaggatccaaacccggagcagcctccagagagcgtgtcgttcacgcggccagcatatgctcagagacctcagaggctcagagacctcagggctggtggtgtggtcggttgtgaccacttgtccctcggaccggctccaggaaccaacctggggaatgtgtgtaggggaagggcgggatagacagtgcccggagcagggaggcgctgaaagacaggaccaagcagcccggccaccagacccgttgtgggaacggaatttcctggcccccagggccacactcgcgtgggaagcatgtcgcggaccctttaaggcgtcatctccctgtctctccgcccccgcctgggacaggcgggacgcccgggacctgacatttggaggctcccaacgtgggagctaaaaatagcagccccgggttactttggggcattgctcctctcccaacccgcgcgccggctcgcgagccgtctcaggccgctggagtttccccggggcaagtacacctggcccgtcctctcctctcagaccccactgtccagacccgcagagtttaagatgcttctgcagcccgggatcctagctggtgggcggagtcctaacacgtgggtgggcggggccttttgttccagggactcttttctcaaaacttcccagtcggaggctggcgggaacccgagaggcgtgtctcgccagccacgcggaggggcgtggcctcattggcccgccccaccaactccagccaaactctaaaccccaggcggagggggcgtggccttctggggtgtgcgggctcctggccaatgggtgctgtgaagggcgtggcccgcgggggcaggagcgaggtggcgggggcttctcgcgtcttttcccccagccccgctccaccagatccgcgggagccccactgctctccgggtccttggcttgtggctgtgggtcccatcgg

SEQ ID NO: 12 - DNA, 18 bp, Artificial Sequence
  ECO RI linkers containg STOP condons in three reading frames
ccggaattctgatagtaa